(19) United States
(12) Patent Application Publication
(10) Pub. No.: US 2012/0040428 A1
(43) Pub. Date: Feb. 16, 2012

(54) PROCEDURE FOR EXTRACTING OF LIPIDS FROM ALGAE WITHOUT CELL SACRIFICE

(76) Inventors: Paul Reep, Ojai, CA (US); Michael Phillip Green, Pacheco, UT (US)

(21) Appl. No.: 13/207,298

(22) Filed: Aug. 10, 2011

Related U.S. Application Data
(60) Provisional application No. 61/373,365, filed on Aug. 13, 2010.

(51) Int. Cl. C12N 13/00; C12M 1/42

(52) U.S. Cl. 435/173.6; 435/283.1; 435/286.1

(57) ABSTRACT

A system and method are disclosed for extracting lipids from algal cells. In the method, lipids are extracted from algal cells by exposing the algal cells in an aqueous medium to an electric field sufficient to cause release of lipids from said cells. In the system, an electric field is formed between two electrodes connected with an electrical power supply and configured such that during use an aqueous medium containing the algal cells passes between the electrodes to extract lipids therefrom.

### RELATED APPLICATIONS

This application claims the benefit of U.S. Provisional Application No. 61/373,365 filed Aug. 13, 2010, entitled PROCEDURE FOR EXTRACTING OF LIPIDS FROM ALGAE WITHOUT CELL SACRIFICE, which is incorporated herein by reference.

### BACKGROUND

The following discussion is provided solely to assist the understanding of the reader, and does not constitute an admission that any of the information discussed or references cited constitute prior art to the present invention.

Microalgae are single celled photosynthetic organisms comprised of proteins, carbohydrates, fats, and nucleic acids in varying proportions. While composition percentage varies among algae species, the lipid content of some species may be up to 50% of their overall mass. When recovered, lipids can be a valuable feedstock for pharmaceutical, nutraceuticals, bio fuel, and foods industries.

Current microalgae lipid extraction methods are designed to dissolve, deconstruct, or fracture the entire cell structure resulting in cell death. The dead biomass, water, and oil, must undergo an energy-intensive separation and drying before lipids can be effectively extracted. Subsequently, more live algae cells are needed to replace the destroyed algal cells. This process can also use harmful chemicals and have a low recovery ratio of lipid to biomass. Because some types of algae microorganisms can be genetically engineered for faster growth rate or higher lipid yields, a live lipid extraction method allowing these organisms to survive the extraction method would be beneficial.

### SUMMARY

This invention relates a live harvest method for extracting lipids from microalgae by applying a suitable electric field to an algae culture. The electrical field stimulates the cells to release a portion of their lipid content which can then be recovered.

A difficulty associated with practical culturing of algae, such as a microalgae culture, for lipids has been extraction of the lipids from the large quantities of biomass. Typically, such extraction has involved processing of the biomass in a manner resulting in substantial destruction of the cells. In contrast, this invention involves live harvest achieved by subjecting live algal cells to suitable electrical stimulation causing release of lipids from the cells. In this method the cells remain viable and thus the same algal culture can continue to grow and produce lipids for subsequent extractions.

Accordingly, a first aspect of the invention concerns a method for extracting lipids from algal cells that involves exposing the cells in an aqueous medium to an electric field sufficient to cause release of lipids from the cells.

Implementations of the invention can include one or more of the following features. The aqueous medium may be a culture medium. The electric field may be a direct current field or an alternating current field, and the electric field may be pulsed. When pulsed, the electric field may be pulsed with a frequency of at least 1 Hz. In some embodiments, the electric field is pulsed with a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz. The algal cells may be exposed to an electric field formed between two electrodes having a voltage of at least 0.5 V applied across said electrodes. The voltage applied across said electrodes can be selected from a group consisting of 0.5 V, 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV. At least 40, 50, 60, 70, 80, 90, 95%, 99%, or 100% of the cells may remain viable following the exposure to an electric field.

Additional embodiments of the method for extracting lipids from algal cells are described in the Detailed Description herein.

In another aspect, a system for extracting lipids from algal cells, wherein the system includes at least two electrodes connected with an electrical power supply and configured such that during use an aqueous medium containing the cells passes between the electrodes to extract lipids therefrom.

Implementations of the invention can include one or more of the following features. In particular embodiments, the at least two electrodes may be mounted in a tank. The tank may have a liquid capacity selected from a group consisting of 11 liters, 15 liters, 20 liters, 30 liters, 50 liters, 60 liters, 75 liters, 100 liters, 150 liters, 200 liters, 250 liters, 300 liters, 400 liters, 500 liters, 750 liters, 1000 liters, 2000 liters, 5000 liters, and 10,000 liters. The electrodes may be whole-tank electrodes, a stacked set of electrodes with gaps between adjacent electrodes (e.g., configured such that medium passes through the plate stack in a sinuous path), or concentrically arranged electrodes. The electrodes may be are mounted in a fluid bypass loop or fluid transfer passageway fluidly connected with the tank for the medium. The power supply electrically connected with the electrodes may apply a voltage across said electrodes of at least 0.5 V. The voltage applied across said electrodes can be selected from a group consisting of 0.5 V, 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV. A pulsed electrical power may be applied across said electrodes. The electrical power may be pulsed at a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz. The electrical power may be supplied to the electrodes as AC power or DC power.

Additional embodiments of the system for extracting lipids from algal cells are described in the Detailed Description herein.

In yet another aspect, a system for extracting lipids from algal cells comprises: at least two electrodes connected with an electrical power supply and configured such that during use an aqueous medium containing the algal cells passes between the electrodes and is exposed to an electric field sufficient to extract lipids therefrom; one or more sensors configured to sense biofeedback data from the aqueous medium; and a computerized control system in electronic communication with the one or more sensors, the computerized control system adjusting the parameters of said electric field based on the biofeedback data received from the one or more sensors. The one or more sensor can be selected from a group consisting of a pH sensor, an oxygen reducing potential (ORP) sensor, a density sensor, a voltage sensor, a current sensor, a conductivity factor sensor, an electrical conductivity sensor, and combinations thereof. The parameters of the electric field may include voltage levels applied between said two electrodes, pulse frequency, or duty cycle on and off times. The one or more sensors may be in fluid communication with the aqueous medium. The power supply electrically connected with the electrodes may apply a voltage across said electrodes of at least 0.5 V. The voltage applied across said electrodes can be selected from a group consisting of 0.5 V, 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV. A pulsed electrical power may be applied across said electrodes. The electrical power may be pulsed at a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz. The electrical power may be supplied to the electrodes as AC power or DC power.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 illustrates a cross-section view of a tank and a set of electrodes, according to a representative embodiment.

FIG. 2 illustrates a cross-section view of a tank and a set of electrodes, according to another representative embodiment.

FIG. 3 illustrates a cross-section view of a bypass pipe and a set of electrodes, according to a representative embodiment.

FIG. 4 illustrates a cross-section, perspective view of set of concentric electrodes, according to a representative embodiment.

FIG. 5 illustrates a cut-away, perspective view of a circular tank having a perimeter wall electrode and a central electrode, according to a representative embodiment.

### DETAILED DESCRIPTION OF THE INVENTION

Various embodiments will be best understood by reference to the drawings, wherein like reference numbers indicate identical or functionally similar elements. It will be readily understood that the components of various embodiments, as generally described and illustrated in the figures herein, could be arranged and designed in a wide variety of different configurations. Thus, the following more detailed description, as represented in the figures, is not intended to limit the scope of the invention as claimed, but is merely representative of embodiments of the invention.

As described in the Summary above, this invention concerns methods, systems, and associated apparatuses for extracting lipids from algae, such as microalgae, without destroying the cells. Instead of killing the cells, the cells are able to tolerate the lipid extraction and remain viable. In general, this is accomplished by exposing the microalgae cells in suspension to a suitable electric field. This causes release of a portion of the cellular lipids that can then be collected from the suspension while the cells remain viable and are able to produce additional lipids that can be recovered in further extraction rounds. For example, in particular cases at least 10%, 20%, 30%, 40%, 50%, 60%, 70%, 80%, 90%, 95%, 99%, or 100% of the previously viable cells remain viable following exposure to the electric field.

At least some aspects of the invention are the result of investigations and considerations of the likely reasons algae are able to forfeit lipids, without permanently harming their cell structure, or affecting organism vigor. For many years, scientists have believed the main purpose algae cells produce and store lipids was to have sufficient energy reserves in lean environments; too little sunlight, insufficient nutrients, etc. Without being bound by any theory, it appears that algae may also produce lipids as a ballasting mechanism for mobility. Lipid, being lighter than water, may allow the algae cells in an open pond or other open body of water to ascend to the surface, thus moving closer to light. On the other hand, when cells need to move away from light, they could descend by releasing lipids. For some species, such movement can be accomplished using cilia or flagella. Those cells, however, represent a group of algae having greater structural complexity than algae currently being studied for oil and fuels production. Many of algal species currently considered for oil production lack the cilia or flagella which provide mobility to move away from predators, move closer or away from light, etc.

Thus, an underpinning approach to at least some of the methods and systems described herein is to stimulate the cell to release lipid into the environment through the cell wall just as they apparently do in nature. Herein a system is described that causes algae cells to release lipids, and do so potentially multiple times without destroying cell viability. In some instances, some or all of the cells continue to thrive, minimizing the need for more/makeup water and nutrients, but are able to continue to uptake CO₂ and produce more lipid in multiple cycles.

Accordingly, in some embodiments, a device or apparatus is used which imposes a pulsed electric field between anode and cathode across an aqueous medium, such as water, containing algal cells, creating an electrical current through that medium. The anode and cathode can be conventional metallic electrodes, whose configuration creates an effective electrical field and/or current within the medium of water and algal cells.

### Electrode Configurations and Placements

Electrodes for applying an electric field can be configured in many different ways. The electrode design and placement should be chosen in conjunction with consideration of factors such as power supply capabilities, power availability, and desired processing capacity.

General examples of electrode configurations include at least three types: 1) whole tank electrodes where all, substantially all, or at least a large fraction of the volume of the tank (herein “tank”) simultaneously has an effective electric field when the electrical supply to the electrodes is activated; 2) by-pass or transfer passage electrodes where electrodes are external to the tank and are situated in a pipe, tube, or other passage for fluid flow; and 3) submerged isolation electrodes where the electrodes are submerged within the tank but are sufficiently electrically isolated from the bulk medium in the tank that at least a large fraction of the current passing between the electrodes follows essentially the shortest path between anode and cathode. With this type of electrode design, the bulk medium in the tank is not exposed to effective electrical field simultaneously. Instead, substantially only the medium between the electrodes is exposed to effective electric field.

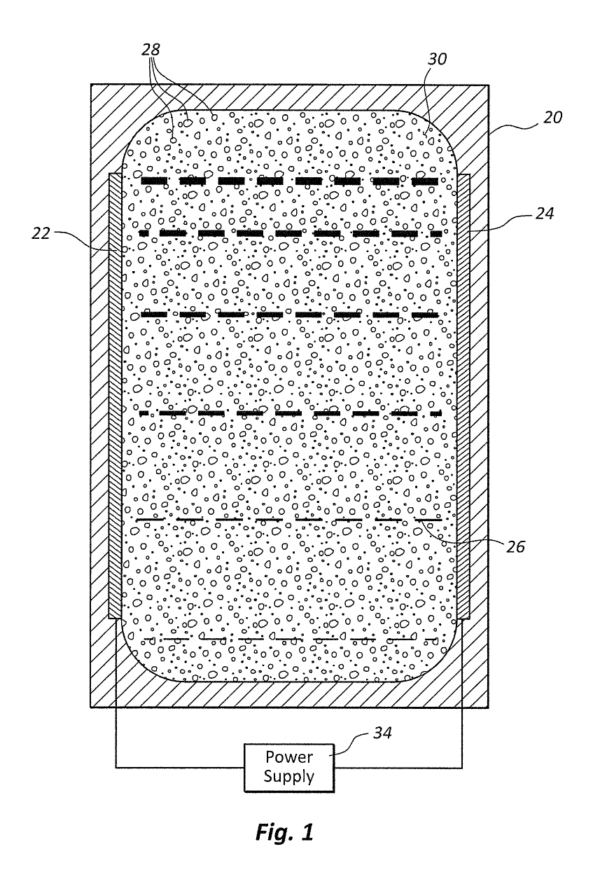

A schematic illustration of a whole tank electrode pair is shown in FIG. 1 as a vertical cross section. The outer lines represent the walls of the tank 20, which define an interior volume 30. The tank 20 used in this and other embodiments may, for example, have an interior volume and a liquid capacity, in liters, of about 1 to 5, 5 to 10, 10 to 20, 20 to 60, 60 to 100, 100 to 150, 150 to 200, 200 to 300, 300 to 500 or greater than 500. Mounted inside two opposing tank walls are the anode 22 and cathode 24 plates respectively. Inside the tank 20 and in contact with both the anode 22 and cathode 24 is the aqueous medium 26 containing algae 28. The plates 22, 24 comprising the electrodes may, for example, have lengths 36 and widths 32 in a ratio of about 1.1:1 to 1.5:1, 1.5:1 to 3:1, 3:1 to 6:1, 6:1 to 10:1, 10:1 to 20:1, or greater than 20:1. The electrodes may be connected to one or more power supplies 34 that supplies power to the electrodes, as described herein.

The electrode set is also configured to allow flow of the medium through the space(s) between the electrodes. For electrode sets, according to some configurations, having more than two electrode plates, such flow can advantageously follow a sinuous path such that the fluid passage across the space between two adjacent electrode plates and then in a substantially antiparallel direction between the next adjacent electrode space. In some embodiments, the set of electrodes includes a stacked set of at least two, three, four, five, six, seven, eight, nine, ten, twenty, thirty or more electrodes, with gaps between adjacent electrodes. When three or more electrodes are used, the electrodes can be configured so that the anode(s) and cathode(s) are equally spaced apart and are alternative. For example, a system with three electrodes can arrange the electrodes in series with an anode, cathode, anode configuration. Alternatively, the three electrodes can include a cathode, anode, cathode configuration. Similarly, with a six-electrode configuration, the electrodes can have an anode, cathode, anode, cathode, anode, cathode configuration.

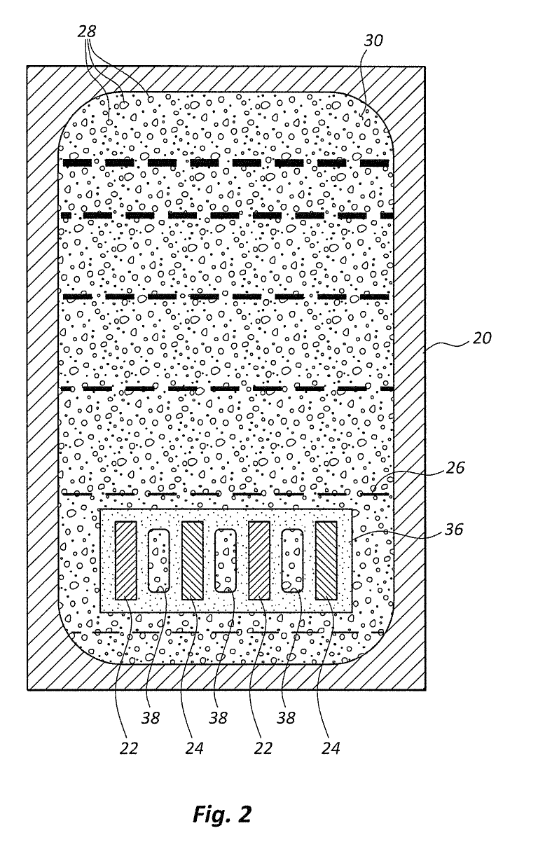

An illustrated example of such an electrode set having more than two electrode plates is depicted in FIG. 2. Other flow designs can also be implemented, e.g., single pass flow across all electrode spaces in the electrode set, radial flow, and diagonal flow. In some configurations, the flow rate may be adjusted in view of the flow pattern to provide adequate residence time for the cells in the electric field for the extraction to effectively occur.

Electrode plate sets of this nature may be installed within a tank 20 or in a bypass loop or transfer passageway. As depicted in FIG. 2, when installed within a tank 20, the electrode set may optionally be configured as a submerged isolation electrode by substantially electrically isolating the electrode plate set from the bulk medium, e.g., by encapsulating the plate electrode set within an electrically insulating housing 36 or similar structure. Medium 26 can then pass through the electrode set by entering through an opening 38 which creates a path having high electrical resistance compared to the electrical resistance between adjacent electrodes (e.g., an inlet with cross sectional area much smaller than the area of the electrode plate and/or an outlet with cross sectional area much smaller than the area of the electrode plate and/or a current path much longer than the current path between adjacent electrodes).

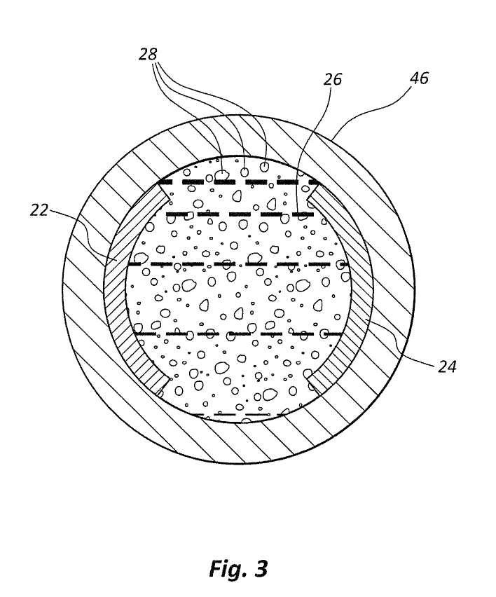

Similar configurations may be used in a bypass loop or transfer passageway, as shown in FIG. 3. That is, fluid enters a sealed plate electrode set through a pipe 46, tubing, or other passageway, follows the designed flow path through the set, and exits through another passageway for return to the tank or to another transfer destination.

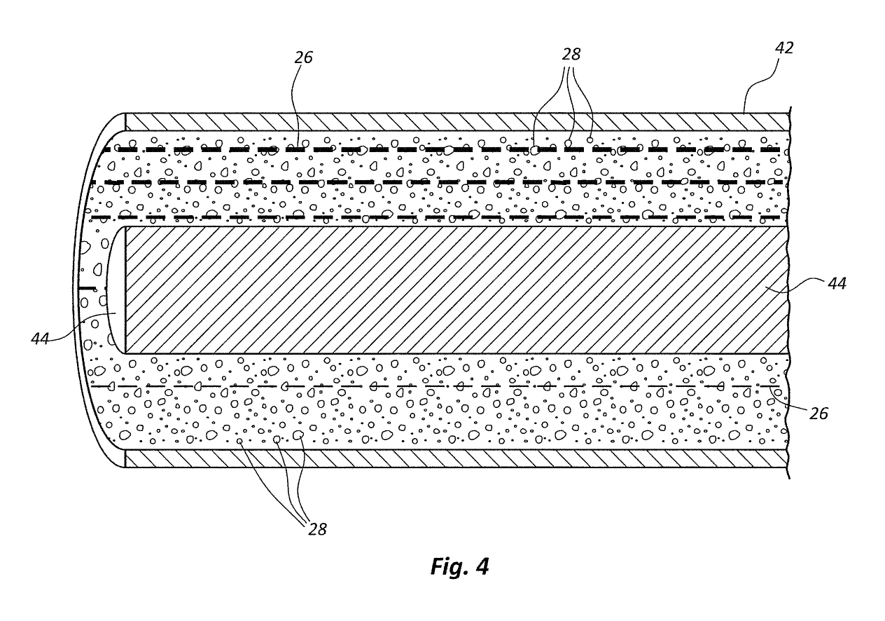

As depicted in FIG. 4, in other embodiments, the electrical field can be applied inline (e.g., when algae culture is pumped through a bypass loop or fluid transfer passageway) rather than applying electrical current in the bulk media. Anode and cathode configurations could include an inner conductive rod 44 or tube and an outer conductive tube 42 internally spaced equally apart which provides a fluid flow pathway between the inside wall of the outer tube and outside wall of the inner rod or tube. The voltage (creating the electric field with resulting electric current) is applied across that space. This spacing additionally provides voltage transfer from the inner rod 40 or tube through the electrical medium to the outer tube 42. This anode and cathode configuration could allow this method to be incorporated as a medium flow conduit.

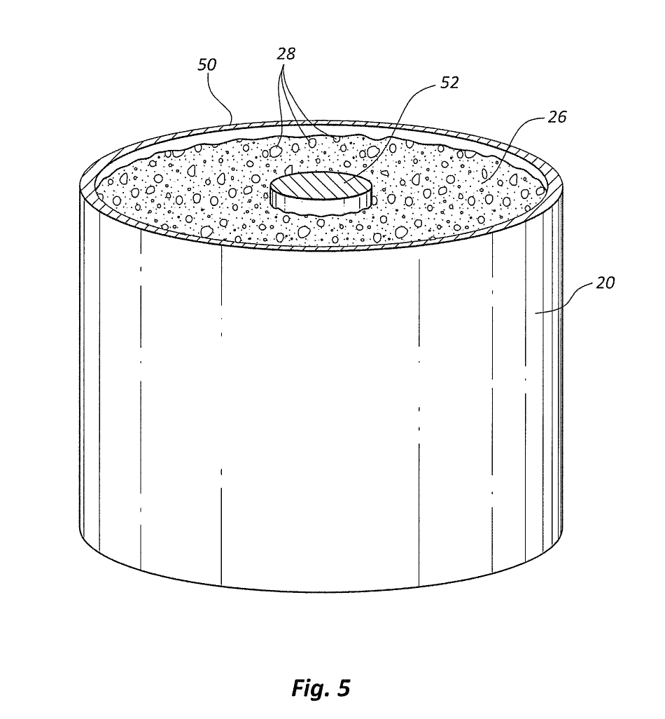

As depicted in FIG. 5, in some embodiments, the electrodes may comprise at least one whole-tank electrode. For example, as shown, a circular tanks 20 allows for the placement of a perimeter wall electrode 50 (e.g., anode) having a preferred size and thickness and a central electrode 52 (e.g., cathode). The central electrode 52 can, for example, be a cylinder such as a rod or tube located in the direct center of the tank such that the central electrode is essentially equidistant from the perimeter wall electrode 50 at all points. This practice allows a voltage to be applied substantially throughout the tank causing current to flow through the aqueous medium 26 between the electrodes 50, 52. In yet other examples, for rectangular tanks anode and cathode 24 can be installed at opposite walls inside the tank 20. As in other cases, the voltage can be applied across those electrodes with resultant electric field and current flow.

Many other electrode configurations can also be utilized, all within the scope of this invention.

### Power Supply and Electrical Field Modulation

For the present methods and associated systems, power supplies provide the electrical power to the electrodes causing lipid release. Any of a variety of different types of power supplies may be chosen, e.g., depending on the particular application, including, for example, electrode configuration, processing capacity, and/or algal strain. In any case, the power supply should provide a desired and adequate voltage between an anode and cathode through the moderate conductivity aqueous medium. Preferred voltages, pulse shapes, and pulse frequencies can depend on the electrical conductivity of the medium and may differ for different algal species or strains.

Many different power supplies can be used for this purpose. In some embodiments, it may be adequate to use uninterrupted direct current (DC) power that may be pulsed, such as using a pulsed electrical input. Any of a large number of DC power supplies is available with a broad range of voltage and amperage capabilities and can be used. DC power supplies can also provide pulsed output, with the pulsing capability being either built into the power supply or incorporated in the circuit as a separate component(s). In some embodiments, the output is programmable, e.g., programmable voltage and/or waveform and/or pulse frequency and/or duty cycle. In many cases, a square wave output or an approximation thereof may be desirable. In some embodiments it may be desirable for the power supply to be designed to handle rapidly switched loads.

Alternatively, an alternating current (AC) power supply can be used, with the frequency and/or voltage of the AC power selected or set at desired levels to provide effective power. The power supply can be designed to provide power at a desired voltage or the voltage can be modulated after the power supply and before the electrical power is delivered to the electrodes. As with DC power, the AC power may be supplied to the electrodes or may be pulsed. In some embodiments it may be desirable for the power to be pulsed; in such embodiments the power supply may be designed to handle rapidly switching loads.

One example of a method of providing power utilizing DC voltage comprises a series of coils which allows a lesser voltage input to be boosted, e.g., into kilovolt (kV) ranges. The frequency of power input to the coil is controlled by a time durational relay circuit utilized for starting and stopping electrical input to the coil. Closing the input allows the coil to electrically charge up and release the higher voltage directed to the cathodes. The usage of voltages within the kilovolt ranges may be based on liquid volumes of the electrode chamber and the conductivity of the bio-liquid environment.

The voltage frequency and the duration of time directed voltage to the primary side of the coil may be controlled utilizing pulse width modulation (PWM). If looking at a series of PWM's on an oscilloscope, sine waves could appear in several different forms. For example, a peak sine wave, (straight up and down) would allow shorter time duration between primary voltage inputs to the coil resulting in a lesser secondary voltage amplification. A longer duration of primary voltage input can be obtained by utilizing a longer duration between the peak's drops down duration. If viewed on an oscilloscope the result would be a plateau (square sine wave) at the top of the peak prior to the sine wave dropping back down. The result is a longer duration of primary voltage to the coil-charge-up time allowing a larger amplification of voltage from the coil's secondary circuit. Further, the length duration of the square sine wave allows the kHz frequency of voltage input to the cathode.

An example of a method utilizing AC voltage comprises a series of step-up transformers that allows a lesser voltage input to be amplified into kilovolt ranges. Utilizing a capacitor inline after the transformers allows further voltage amplification due to its ability to store voltage and release this higher voltage upon reaching the capacitor's storage limits. Voltage produced is directed to the cathode. In reference to AC voltage, unless otherwise indicated the voltage is RMS (root mean square) voltage.

AC voltage produces its own PWM in the form of Hz cycles with AC always appearing on an oscilloscope in a waveform. AC can be altered by changing frequency. In many cases the AC frequency will be normal line frequency, e.g., about 50 to 60 Hertz (Hz), but may be higher or lower. The number of cycles per second desired can be modified based on the density of the electrical medium within the tank. AC power will most often be provided having typical sine waveform, but can also be provided in other forms, e.g., square wave.

The voltage utilized can depend on a variety of factors, e.g., on the configuration of the electrodes, the electrical conductivity of the medium, the power pulse regime selected, and/or the algal strain. For example, in some cases, the voltage (AC or DC) will be 0.5 to 15 volts (V), 15 to 75 V, 75 to 250 V, 250 to 1000 V, 1 to 2 kilovolts (kV), 2 to 5 kV, 5 to 20 kV, 20 to 50 kV, or even higher.

In some cases, it is desirable to have comparatively low current, i.e., low amperage (A). Thus, for example, the current through the medium may be 50 to 200 milliamp (mA), 200 to 400 mA, 400 to 600 mA, 600 to 1000 mA, or 1 to 5 A, but in some cases the current may be higher.

As indicated, in some configurations, it can be desired to provide pulsed power (AC or DC) to cause algal cells to release lipids. To pulse power, the frequency of pulsing can be varied as can the duty cycle. In this context, the term duty cycle refers to the relative lengths of the on and off portions of each power cycle, and can be expressed, for example, as a ratio of the duration of the on portion of the cycle to the total time for the cycle or as a ration of the duration of the on portion of the cycle to the duration of the off portion of the cycle or by stating the on and off durations or by stating either the on or off duration and the total cycle duration. Unless otherwise indicated or is clear from the context, duty cycle will be stated herein as the ratio of on duration to off duration for a cycle.

In some cases, the power pulse frequency will be 1 to 10 Hertz (Hz), 10 to 50 Hz, 50 to 100 Hz, 100 to 200 Hz, 200 to 500 Hz, 500 to 1000 Hz, 1 to 2 kilohertz (kHz), 2 to 5 kHz, 5 to 10 kHz, 10 to 20 kHz, or 20 to 40 kHz. For any of the pulse frequencies just indicated or other frequency used, the duty cycle may, for example, be in a range of 0.1 to 0.2, 0.2 to 0.3, 0.3 to 0.5, 0.5 to 1, 1 to 1.2, 1.2 to 1.5, 1.5 to 3, 3 to 5, or 5 to 10 (expressed as the ratio of on duration to off duration).

The duration of treatment time needed for effective harvest can vary depending on factors such as the algal strain and electrical stimulation conditions. In particular cases, the stimulation duration, i.e., the residence time, in minutes, for cells within a treatment zone, is 0.01 to 0.1 to 0.3, 0.3 to 0.5, 0.5 to 1, 1.0 to 2, 2 to 5, or 5 to 10, or greater than 10.

### Process Control

While it is practical to operate the lipid harvest apparatus manually, it may be desirable to at least partially automate the system. Thus, sensors can be located within the growth tank to relay bio-feedback information on selected parameters to a control system, usually a computerized control system, programmed to take those culture feedback parameters and appropriately control the harvest system.

Thus, in some configurations, a computerized control system controls the initiation and termination of harvest and/or process parameters such as the voltage and/or energy level, pulse frequency, on and off times, and/or the length of each individual duty cycle. The computerized control system may regulate and adjust these controls based on biofeedback information received from various sensors of the system, such as pH sensors, oxygen reducing potential (ORP) sensors, density sensors, voltage or current sensors, conductivity factor sensors, and electrical conductivity sensors. For example, the computerized control system can increase the pulse frequencies, for example, when elevated levels of biomass density are detected. Similarly, the system may lower the pulse frequencies when a lower density culture is detected.

In some embodiments, a dynamic power control module (DPC) is incorporated into the computerized control system. The DPC can be comprised of a series of sensors tied back to a main computer control unit, or central processor. This module can interface with existing industrial control systems and/or run stand-alone. The DPC can take feedback from pH, turbidity, oxygen reduction potential (ORP), conductivity, resistance, temperature, and/or other sensors as biofeedback. Using algorithms appropriate for the culture and desired product, the system calculates when the optimum harvest cycle should or will occur. This harvest cycle may be based, for example, on the desired output from the algae, the algae species, the geographic region of the algae growth plant, and/or many other factors. Once the DPC has calculated that the culture is ready, it will initiate a harvest sequence. The DPC will then control the power output in the form of pulse functions, frequencies and other output determiners mentioned above, for optimal harvest of that dynamic batch of algae culture.

The present lipid extraction systems may be used with or incorporated with any of a large variety of algal growth systems, including but not limited to systems such as those described in Fraser et al., U.S. Provisional Application No. 61/220,629 and the patents and patent applications cited therein, all of which are incorporated herein by reference in their entireties. The present extraction or harvest system may be used instead of or in addition to lipid extraction processes, apparatus, or systems described therein.

### EXAMPLES

### Example 1

### Small Scale Harvest Prototype

A test prototype of an apparatus capable of creating an electrical field for live harvest includes a Petri dish within which are mounted an anode and a cathode. This prototype showed the capacities of passing a DC voltage discharge from a submerged anode through water containing algae cells to the cathode. Using a small DC discharge at 12.5 V with minimal current for a time of three minutes resulted in the release of a visible oily substance on the surface of the water. Microscope inspection of the water containing algae biomass showed minor cell wall fracturing associated with the oily substance release, but the absence of cell flocculation, which commonly occurs during an electrolysis process when substantial amperage is applied. When cell flocculation is inspected under a microscope, individual cells show major fracturing, similar to a slice of pie being removed from the cell organism, which results in cell death.

This particular biomass sample was preserved and re-inspected several days later. When inspected under the microscope the sample showed no signs of cell flocculation or cell wall damage and the individual cells appeared normal and healthy.

### Example 2

### Medium Scale Dry Cell & Coil Pulse Prototype

In another test prototype, a series of stainless steel plates having a minimum thickness of 0.022 inches. This series was located and suspended within a tank containing an aqueous microalgae culture. The water provided a moderately electrically conductive medium allowing a current to pass between appropriately spaced suspended plates. The culture was introduced into the feed plate, and traveled through the series of plates while undergoing electrical stimulation at 12 V, very low amperage, and at 10 to 30 Hz frequency. Again, an oily substance was visible on the surface of the water. The sample of the algae biomass were drawn and inspected under a microscope, no visible signs of flocculation were present and biomass cells appeared in good condition.

### Example 3

### Medium Scale Harvest Prototype

A third test prototype of an apparatus was constructed that included a tank with a capacity of up to nine liters of the water containing an algae biomass. A series of stainless steel plates having a minimum thickness of 0.022″ was located and suspended within the tank. Twelve volts DC was applied to the series of anode and cathode plates for a period of ten minutes. Once again, an oily substance appeared on the surface of the water indicating a release of lipids from the biomass and the absence of any biomass flocculation. Inspection under the microscope further indicated minor fracturing of the cell wall without sections of the cell being removed or fracturing traveling to the cell's nucleus. A series of tests were conducted using 12 VDC as the constant voltage input, absent of any amperage current. The 12 V constant was pulsed at several different Hz frequencies to the anode, (electrical input frequency was introduced and disrupted on the negative side of the anode/cathode circuit). It was discovered when the biomass density was increased from three grams per liter to ten grams per liter, and that an increase in pulse frequencies resulted in an improved harvest yield.

A second series of tests were conducted with the third test prototype. In this series of tests, a 12V DC coil was introduced into the electrical system. The coil was used to boost the input voltage to the tank by way of the anode and cathode plates. Voltage and current testing was performed on the coil determined that the coil had an output of 40 kV and very low current. Using a pulse width modulator allowed the adjustment of the frequency of electrical input to the coil circuit with the secondary coil circuit connected to the anode plate. Frequencies of voltage input in the 10 to 30 Hz range showed an increased in oily substance floating on the medium. Again, samples of the algae biomass were drawn and inspected under a microscope, no visible signs of flocculation were present and biomass cells appeared in good condition.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims. It will be readily apparent to one skilled in the art that varying substitutions and modifications may be made to the invention disclosed herein without departing from the scope and spirit of the invention. For example, variations can be made to the design, size, and placement of electrodes as well. Thus, such additional embodiments are within the scope of the present invention and the following claims.

The invention illustratively described herein suitably may be practiced in the absence of any element or elements, limitation or limitations, which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. Also, unless indicated to the contrary, where various numerical values or value range endpoints are provided for embodiments, additional embodiments are described by taking any two different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

1. A method for extracting lipids from algal cells, comprising exposing said cells in an aqueous medium to an electric field sufficient to cause release of lipids from said cells.

2. The method of claim 1, wherein said electric field is a direct current field.

3. The method of claim 1, wherein said electric filed is an alternating current field.

4. The method of claim 1, wherein said electric field is pulsed.

5. The method of claim 1, wherein said electric field is pulsed with a frequency of at least 1 Hz.

6. The method of claim 5, wherein said electric field is pulsed with a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.

7. The method of claim 1, wherein exposing said cells to an electric field includes introducing said cells between two electrodes having a voltage of at least 0.5 V applied across said electrodes.

8. The method of claim 7, wherein exposing said cells to an electric field includes introducing said cells between two electrodes having a voltage selected from a group consisting of 0.5 V, 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV applied across said electrodes.

9. A system for extracting lipids from algal cells, comprising at least two electrodes connected with an electrical power supply and configured such that during use an aqueous medium containing said algal cells passes between said electrodes to extract lipids therefrom.

10. The system of claim 9, the system further comprising a tank having a liquid capacity of at least 10 liters.

11. The system of claim 10, where the liquid capacity of the tank is selected from a group consisting of 11 liters, 15 liters, 20 liters, 30 liters, 50 liters, 60 liters, 75 liters, 100 liters, 150 liters, 200 liters, 250 liters, 300 liters, 400 liters, 500 liters, 750 liters, 1000 liters, 2000 liters, 5000 liters, and 10,000 liters.

12. The system of claim 9, wherein said at least two electrodes comprises a stacked set of at least three electrodes with gaps between adjacent electrodes.

13. The system of claim 9, wherein said electrodes are mounted in a fluid bypass loop or fluid transfer passageway fluidly connected with a tank for said medium.

14. The system of claim 9, wherein said electrodes are concentrically arranged.

15. The system of claim 9, wherein the power supply electrically connected with said electrodes applies a voltage across said electrodes of at least 0.5 V.

16. The system of claim 15, wherein the power supply electrically connected with said electrodes applies a voltage across said electrodes selected from a group consisting of 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV applied across said electrodes.

17. The system of claim 9, wherein a pulsed electrical field is applied across said electrodes.

18. The system of claim 17, wherein said electrical power is pulsed at a frequency of at least 1 Hz.

19. The system of claim 18, wherein said electrical power is pulsed at a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.

20. The system of claim 9, wherein said aqueous medium is culture medium.

21. A system for extracting lipids from algal cells, the system comprising:
at least two electrodes connected with a power supply and configured such that during use an aqueous medium containing said algal cells passes between said electrodes and is exposed to an electric field sufficient to extract lipids therefrom;
one or more sensors configured to sense biofeedback data from said aqueous medium; and
a computerized control system in electronic communication with said one or more sensors, the computerized control system adjusting the parameters of said electric field based on said biofeedback data received from said one or more sensors.

22. The system of claim 21, wherein said parameters of said electric field include at least one of voltage levels applied between said two electrodes, pulse frequency, and duty cycle on and off times.

23. The system of claim 21, wherein said one or more sensors are in fluid communication with said aqueous medium.

24. The system of claim 21, wherein the one or more sensor are selected from a group consisting of a pH sensor, an oxygen reducing potential (ORP) sensor, a density sensor, a voltage sensor, a current sensor, a conductivity factor sensor, an electrical conductivity sensor, and combinations thereof.

25. The system of claim 21, wherein the power supply electrically connected with said electrodes applies a voltage across said electrodes of at least 0.5 V.

26. The system of claim 25, wherein the power supply electrically connected with said electrodes applies a voltage across said electrodes selected from a group consisting of 1 V, 2 V, 3 V, 5 V, 10 V, 15 V, 20 V, 30 V, 40 V, 50 V, 75 V, 100 V, 250 V, 1 kV, 2 kV, 5 kV, 10 kV, 20 kV, and 50 kV applied across said electrodes.

27. The system of claim 21, wherein a pulsed electrical field is applied across said electrodes.

28. The system of claim 27, wherein said electrical power is pulsed at a frequency of at least 1 Hz.

29. The system of claim 28, wherein said electrical power is pulsed at a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.
